(19) United States
(12) Patent Application Publication
(10) Pub. No.: US 2023/0157233 A1
(43) Pub. Date: May 25, 2023

(54) METHODS FOR IMPROVED MICROSPORE EMBRYOGENESIS AND PRODUCTION OF DOUBLED HAPLOID MICROSPORE-DERIVED EMBRYOS

(71) Applicant: Monsanto Technology LLC, St. Louis, MO (US)

(72) Inventors: Huachun Wang Larue, Chesterfield, MO (US); Huai Wang, Chesterfield, MO (US)

(21) Appl. No.: 17/921,900

(22) PCT Filed: Jan. 26, 2021
(86) PCT No.: PCT/US2021/014998

§ 371 Date: Oct. 27, 2022

Related U.S. Application Data
(60) Provisional application No. 63/019,150, filed on May 1, 2020.

(51) Int. Cl. A01H 4/00; A01H 1/08

(52) U.S. Cl. CPC A01H 4/005; A01H 1/08

(57) ABSTRACT

The invention provides novel methods for microspore embryogenesis and the production of doubled haploid embryos. For example, the methods provided include obtaining a plurality of flower buds from a donor plant, determining the developmental stage of microspores comprised within said flower buds, selecting flower buds comprising microspores at a desired developmental stage, treating said flower buds, isolating microspores from said flower buds, and culturing said microspores in induction medium or treating said isolated microspores with a chromosome doubling agent.

### REFERENCE TO RELATED APPLICATION

This application claims the benefit of U.S. Provisional Application Ser. No. 63/019,150, filed May 1, 2020, which is herein incorporated by reference in its entirety.

### FIELD OF THE INVENTION

The present invention relates to the field of plant breeding and agricultural biotechnology. More specifically, the invention provides methods for canola and oilseed rape microspore embryogenesis and doubled haploid production.

### BACKGROUND OF THE INVENTION

The use of doubled haploids (DH) in a plant breeding program allows new inbred populations to be created from desired parents in 1-2 generations. Haploids (1n) contain only one set of chromosomes that are found after meiosis in male or female gametes. Doubled haploids (2n) carry two identical sets of chromosomes that were derived from haploids. Unlike ordinary diploids that achieve 2n chromosomes through fertilization between male and female gametes, DH become diploid through chromosome doubling of the haploid chromosome by chemical or spontaneous means. Utilization of DH can enable breeding to obtain pure 2n homozygous plants in a single generation, compared to 6 or more generations of selfing or backcrossing in typical breeding schemes. It is a critical tool to reduce breeding cycle time with improved heritability to accelerate genetic gain. In addition, DH is also a useful tool to obtain homozygous plants faster in other processes, such as gene stacking, genome editing, cytoplasm and nuclear genome exchange, trait integration, etc. (Ren et al. 2017).

Canola/oilseed rape DH production mainly relies on a microspore culture-based process which consists of many steps, from donor Fi plant care, bud preparation, microspore isolation, microspore-derived embryogenesis, and microspore-derived embryo germination and plant regeneration. Microspores that are produced from Fi plants have already gone through meiosis and genetic recombination. DH derived from microspores of Fi plants are segregating for different traits and are therefore ideal for breeding selection. Despite intensive research efforts to improve the canola and oilseed rape DH process over the past few decades, the success of microspore culture-based DH production appears to be highly genotype-dependent. Many canola and oilseed rape genotypes, especially those that are relevant to commercial breeding, tend to be recalcitrant to microspore culture DH processes and render the DH production outcomes as highly variable and unpredictable. There were previous efforts in identifying genetic loci associated with microspore embryogenesis potential (Ecke et al. 2015). Although such loci can be introgressed into recalcitrant breeding lines, it is a less than practical approach in practice, due to high costs and the potential for undesirable genetic loads and yield drag associated with introgressions.

For microspore culture-based DH to be of value to a breeding program, the most important metric is the efficiency of embryogenesis. However, improving embryogenesis efficiency alone does not necessarily lead to an increase in doubled embryos. While there are many known processes that increase embryogenesis efficiency, these processes are of little or no value to a breeding program if only a very small percentage of the embryos have doubled chromosomes. Here, the present inventors introduce the novel concept of “doubled embryogenesis” which is a measurement of both embryogenesis efficiency and chromosome doubling rate during the process of turning microspores into doubled embryos.

The present inventors have developed a novel method, which is described herein, that enables a step-change improvement over current methods of canola/oilseed rape microspore culture-based DH production. This novel method delivers consistent improvement in the production of doubled microspore-derived embryos across a wide range of genotypes, including pedigrees from canola and winter oilseed rape, female and male-heterotic groups, and inbred and hybrid lines. Surprisingly, with the novel combination of a cold pretreatment followed by colchicine treatment of microspores, a dramatic improvement in doubled microspore-derived embryogenesis efficiency and response consistency was observed. This improvement has not been previously described. Based on this discovery, a new DH process was developed to incorporate the “cold-colchicine” treatment, which combines cold pretreatment of buds/microspores followed by colchicine treatment of microspores. This serves to deliver significant time and resource savings.

In summary, the new method comprises the following major innovations: 1) precise staging for selection of microspores at LLU-EBC stage; and 2) novel cold-colchicine combination treatment of selected microspores for doubled embryogenesis. These steps result in more efficient and consistent doubled embryo productions compared to previous methods which enables redesign of the current DH pipeline workflow into a more efficient process with shortened cycle time.

### SUMMARY OF THE INVENTION

In one aspect of the present disclosure, a method for producing embryos from microspores is provided comprising the steps of: a) obtaining a plurality of flower buds from a donor plant; b) determining the developmental stage of microspores comprised within said flower buds; c) selecting flower buds comprising microspores at a desired developmental stage; d) treating said flower buds; e) isolating microspores from said flower buds; and f) culturing said microspores in induction medium, thereby producing embryos. In one embodiment, of the method, the desired developmental stage is defined as the late late-uninucleate stage or the early bi-cellular stage. Optionally, the developmental stage can be determined by nuclear staining of said microspores. In specific embodiments, the treating comprises incubating said flower buds at a temperature of about 2° C. to about 8° C. for about 24 hours to about 72 hours. In other embodiments, the incubating is carried out for about 24 hours to about 48 hours. In still further embodiments, culturing is carried out at a density of about 2.0×10⁴ to about 1.0×10⁵ microspores/mL. In even further embodiments, culturing is carried out at a density of about 2.0×10⁴ microspores/mL, about 3.0×10⁴ microspores/mL, about 4.0×10⁴ microspores/mL, about 5.0×10⁴ microspores/mL, about 6.0×10⁴ microspores/mL, about 7.0×10⁴ microspores/mL, about 8.0×10⁴ microspores/mL, about 9.0×10⁴ microspores/mL, or about 1.0×10⁵ microspores/mL. Examples of donor plants include canola, cauliflower, broccoli, pepper, cabbage, soybean, cotton, or corn plants.

In another aspect, the present disclosure provides a method for producing potentiated microspores comprising the steps of: a) obtaining a plurality of flower buds from a donor plant; b) determining the developmental stage of microspores comprised within said flower buds; c) selecting flower buds comprising microspores at a desired developmental stage; d) pretreating said flower buds; e) isolating microspores from said flower buds; and t) treating said isolated microspores with a chromosome doubling agent to produce potentiated microspores. In one embodiment, the desired developmental stage is defined as the late late-uninucleate stage or the early bi-cellular stage. In another embodiment, the developmental stage is determined by nuclear staining of said microspores. In yet another embodiment, the pretreating comprises incubating said flower buds at a temperature of about 2° C. to about 8° C. for about 24 hours to about 72 hours. The incubating may be carried out, for example, for about 24 hours to about 48 hours. In specific embodiments, culturing is carried out at a density of about 2.0×10⁴ to about 1.0×10⁵ microspores/mL. In even further embodiments, culturing is carried out at a density of about 2.0×10⁴ microspores/mL, about 3.0×10⁴ microspores/mL, about 4.0×10⁴ microspores/mL, about 5.0×10⁴ microspores/mL, about 6.0×10⁴ microspores/mL, about 7.0×10⁴ microspores/mL, about 8.0×10⁴ microspores/mL, about 9.0×10⁴ microspores/mL, or about 1.0×10⁵ microspores/mL. In another embodiment, the chromosome doubling agent is colchicine. Optionally, colchicine can be used at a concentration of about 25 mg/L to about 1600 mg/L. In some embodiments, treating comprises applying colchicine at a concentration of about 100 mg/L to about 1000 mg/L or at a concentration of about 500 mg/L to about 1000 mg/L. In specific embodiments, treating comprises incubating said microspores at a temperature of about 32° C. for a duration of about 24 hours to about 72 hours. In other embodiments, the incubating is carried out for about 24 hours to about 48 hours or for about 40 hours to about 48 hours. In further embodiments, the method for producing microspores comprising doubled chromosomes further comprises the steps of: g) culturing said microspores to obtain at least a first embryo; and h) regenerating a doubled haploid plant from said embryo. Examples of donor plants include canola, cauliflower, broccoli, pepper, cabbage, soybean, cotton, or corn plants.

In another aspect, the present disclosure provides a method producing doubled haploid embryos from microspores comprising the steps of: a) providing microspores at a desired developmental stage; b) pretreating said microspores under cold conditions for a fixed period of time; c) treating said microspores in a medium containing an effective concentration of colchicine to induce chromosome doubling; d) culturing said treated microspores of step c) in induction medium, thereby producing embryos; and e) recovering doubled haploid embryos from said induction medium. In some embodiments, the cold conditions comprise a temperature of about 0° C. to about 25° C. In specific embodiments, the cold conditions comprises a temperature of about 2° C. to about 8° C. In some embodiments, the fixed period of time is about 12 hours to about 72 hours. In further embodiments, the fixed period of time is about 24 hours to about 48 hours. In other embodiments, the effective concentration of colchicine is about 25 mg/L to about 1600 mg/L in the medium. In further embodiments, the effective concentration of colchicine is about 200 mg/L to about 1000 mg/L in the medium. In yet further embodiments, the effective concentration of colchicine is about 500 mg/L to about 1000 mg/L in the medium. In some embodiments, step c) of the method is carried out at a fixed temperature for a fixed amount of time. In other embodiments, step c) is carried out at 32° C. for about 12 hours to about 72 hours. In further embodiments, step c) is carried out at 32° C. for about 24 hours to about 48 hours.

In yet another aspect, the present disclosure provides a method for producing doubled haploid Brassica embryos comprising the steps of: a) providing Brassica microspores at or between the late late-uninucleate and early bi-cellular developmental stages; b) pretreating said microspores under cold conditions for a period of about 12 hours to about 72 hours; c) treating said microspores in a medium containing an effective concentration of colchicine to induce chromosome doubling; d) culturing said treated microspores of step c) in induction medium, thereby producing embryos; and e) recovering doubled haploid Brassica embryos from the induction medium. In some embodiments, the cold conditions comprise temperatures of 2° C. to about 8° C. In other embodiments, the effective concentration of colchicine to induce chromosome doubling is about 25 mg/L to about 1600 mg/L in the medium. In specific embodiments, the effective concentration is about 200 mg/L to about 1000 mg/L in the medium. In further embodiments, the effective concentration of colchicine is about 500 mg/L to about 1000 mg/L in the medium.

### BRIEF DESCRIPTION OF THE DRAWINGS

FIG. 1: Shows a schematic of main steps in the canola/oilseed rape doubled haploid (DH) workflow. Inconsistent microspore embryogenesis and low doubling rates of generated embryos are two of the major bottlenecks that limit the DH efficiency in the current canola/oilseed rape DH production process.

FIG. 2: Shows images of bud length measurements and microspore staging using DAPI staining in two canola lines and one oilseed rape line. Buds having lengths of 2.5 mm, 3.0 mm, 3.5 mm, 4.0 mm, 4.5 mm, and 5.0 mm were selected for staging. Buds containing microspores at the late uninucleate (LU) stage span a wider range (up to 1.5 mm) in length than other stages (indicated by the thinner bar in the figure). Microspores at the late late-uninucleate (LLU) stage contain a single nucleus that appears stretched, as opposed to the normal rounded morphology. Microspores at the early bi-cellular (EBC) stage comprise two formed nuclei that are bound together and both relatively faint in color and large in size. Microspores staged in between the LLU-EBC stages are indicated by the thicker bar in the figure.

FIGS. 3A, 3B and 3C: Early late-uninucleate (ELU) and LLU-EBC staged microspores were isolated and cultured to induce embryogenesis. Four weeks after induction, embryos were selected for a ploidy assay. The doubled embryogenesis from the LLU-EBC stage is consistently higher than embryos derived from the ELU stage, even though ELU stage yield significantly higher rate of embryogenesis. 3A: Shows embryo count produced from one million microspores. 3B: Shows doubling efficiency measured as a percentage of embryos that have undergone chromosome doubling after colchicine treatment. 3C: Shows doubled embryo count from one million microspores.

FIGS. 4A and 4B: Microspores from canola line 57777 were isolated, split into groups that were treated with either 200 mg/L, 600 mg/L, or 1000 mg/L colchicine for 24 hours or 48 hours, and cultured to induce embryogenesis. Embryos were selected for a ploidy assay 4 weeks after induction. Embryo doubling rate increased with increasing concentrations of colchicine. However, at all three concentrations shown here, embryos derived from microspores treated with colchicine for 48 hours showed a greater than 2 fold increase in doubled embryogenesis compared to those treated for 24 hours. 4A: Shows doubling efficiency measured as the percentage of embryos having undergone chromosome doubling after colchicine treatment. 4B: Shows doubled embryogenesis which is measured by doubled embryo count per million microspores.

FIGS. 5A, 5B, and 5C: 5A: Illustrates the experimental design of comparison experiments where cold-colchicine treatment is evaluated against different canola/oilseed rape DH methods that have been previously reported. Staged buds were collected and split for non-cold or cold treatment, then isolated microspores were split again for colchicine treatment respectively. 5B and 5C: Show doubled embryo production data from four different treatment conditions for canola female line 57777 and male line CD6113, respectively.

### DETAILED DESCRIPTION

Doubled haploid (DH) plants are a valuable tool to plant breeders, particularly for generating inbred lines. A great deal of time is spared as homozygous lines are essentially generated in a single generation, negating the need for multigenerational conventional inbreeding, thus accelerating breeding genetic gain. In addition, DH plants are entirely homozygous, thus there is no allele masking effect between genotype and phonotype. They are very amenable to breeding selection and quantitative genetics studies. For breeders, DH populations have been particularly useful in QTL mapping, cytoplasmic conversions, and trait introgression.

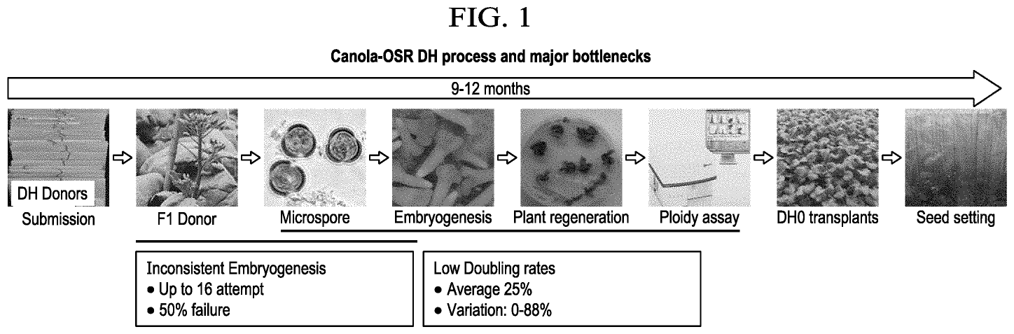

Canola/oilseed rape DH production is a microspore culture-based process which consists of many steps and takes up to 9-12 months with current protocols (FIG. 1). Although many improvements have been made over the last few decades, it is still a relatively low efficiency and highly variable process that leads to unpredictable production outcomes. The present invention improves the canola/oilseed rape DH process and leads to a dramatic increase in the production of doubled haploid embryos. Furthermore, this system can be used for microspore culture-based DH processes in other species, such as broccoli, pepper, cauliflower, wheat, rice, soybean, cotton, corn, etc. In addition, the cold-colchicine combination treatment may be used to increase microspore culture-based embryogenesis in other plant species, or other general tissue culture and transformation processes that involve embryogenesis. This microspore culture system could also be useful as a platform in genome editing and gene and protein functional studies.

This invention delivers step-change improvement in reliability and efficiency for microspore embryogenesis and chromosome doubling in embryos by eliminating the need for a ploidy assay of regenerated seedlings, which is one of the most tedious and costly steps in the current canola/oilseed rape DH process. This invention therefore can significantly improve the efficiency of a DH breeding program by scaling up the volume in the production pipeline without additional investment.

### Embryogenesis and Doubled Haploids

Microspore-derived embryogenesis is a unique process in which haploid, immature pollen (microspores) are induced by one or more stress treatments to form embryos in culture. In more general aspects, the invention presents a new methodology for the induction of embryogenesis in canola/oil rapeseed microspores which comprises subjecting the compositions containing microspores to a stressor. The protocol consists of pretreating a plant composition, for example, organs such as flower buds containing microspores, under conditions which divert the microspores from gametophytic development to that of embryogenic development. The pretreatment includes incubation of the plant composition, preferably flower buds, at a cold temperature which is a stress factor. Microspores are then isolated from the buds in an isolation medium capable of maintaining microspore viability and embryogenic potential. These isolated microspores are then exposed to an embryoid/callus promoting medium (such as NLN medium) that includes 6-BAP in a range of 0.01-0.5 mg/L and cefotaxime at 100 mg/L, as an antimicrobial agent (Nitsch and Nitsch, 1967; Lichter, 1982; Charne and Beversdorf, 1988). The source of nitrate in this medium is potassium nitrate while L-glutamine, L-serine, and glutathione serve as sources for amino acids.

Although microspore-containing plant organs such as anthers can generally be pretreated at any cold temperature below about 18° C., a range of about 2° C. to about 8° C. is preferred. Although other temperatures yield embryoids and regenerated plants, cold temperatures produce optimum response rates compared to pretreatment at temperatures outside the preferred range. Microspores contained in plant material (such as isolated buds, inflorescences, or whole plants) or isolated microspores are typically pretreated for a predetermined amount of time, preferably for about 24 hours to about 72 hours. However, other amounts of time for pretreatment are within the scope of this invention, as long as isolated microspores will undergo embryogenesis. The response rate is measured as either as the number of total embryos or the number of doubled embryos per number of microspores initiated in culture. Exemplary methods of microspore culture are disclosed in, for example, U.S. Pat. Nos. 5,322,789 and 5,445,961, the disclosures of which are specifically incorporated herein by reference. The inventors have identified a preferred culturing density of about 2.0×10⁴ to about 1.0×10⁵ microspores/mL. It is understood that the microspore culture density may be adjusted based on the species being cultured.

As used herein, the term “tissue culture” indicates a composition comprising isolated cells of the same or a different type or a collection of such cells organized into parts of a plant. Exemplary types of tissue cultures are protoplasts, calli, and plant cells that are intact in plants or parts of plants, such as embryos, pollen, flowers, leaves, roots, root tips, anthers, and the like. In a preferred embodiment, the tissue culture comprises microspores, embryos, protoplasts, meristematic cells, pollen, leaves or anthers derived from immature tissues of these plant parts. Means for preparing and maintaining plant tissue cultures are well known in the art (U.S. Pat. Nos. 5,538,880; and 5,550,318, each incorporated herein by reference in their entirety). By way of example, a tissue culture comprising organs such as anthers has been used to produce regenerated plants (U.S. Pat. Nos. 5,445,961 and 5,322,789; the disclosures of which are incorporated herein by reference).

Pollen development is known to be divided into different stages. Microspores refer to a collection of cells in stages after pollen mother cells undergo meiosis. After releasing from the tetrad structure until fully matured pollen, microspores are further defined as uninulceate, bicelluar and tricellular, based on the nuclei counts in the cell. For the uninucleate stage, the microspores only contain one nucleus. The uninucleate stage can be further divided into the early-, mid-, and late-uninucleate stages. These stages have been well described and illustrated previously (Fletcher et al., 1998; Kott et al., 1988). For anther/microspore culture, if buds are the plant composition, they are preferably selected at a stage when the microspores are at the uninucleate (that is, include only one, rather than 2 or 3 nuclei) or early bicellular stage. Methods to determine the correct stage are well known to those skilled in the art and include mitramycin and 4′-6-diamidino-2-phenylindole (DAPI) fluorescent staining, trypan blue, and acetocarmine squashing. The late-uninucleate (LU) microspore stage was previously believed to be the developmental stage most responsive to embryogenesis and subsequent plant production. The present invention further divides the LU stage into the early LU (ELU) and late LU (LLU) stages and shows that microspores in the late late-uninucleate stage (LLU) to early bi-cellular (EBC) stage are highly responsive to embryogenesis, though not as responsive as microspores in the ELU stage. Microspores in the LLU to EBC stage are however more responsive to chromosome doubling when compared to microspores in the ELU stage. As a result, using microspores in the LLU-EBC stages will result in a high number of embryos that are predominantly DH embryos. This is advantageous in DH production for breeding purposes. A protocol utilizing DAPI (4′,6-diamidino-2-phenylindole) DNA staining was developed by the present inventors to precisely identify microspores at these stages. This protocol enabled cells staged as the LU stage to be further divided into the early late-uninucleate (ELU) and late late-uninucleate (LLU) stages.

Microspore-derived haploid embryos can be converted to doubled haploids by chromosome doubling agents and/or through spontaneous doubling. In one aspect, a method of chromosome doubling provided herein comprises the use of colchicine. Colchicine may be used at concentrations of about 25 mg/L to about 1600 mg/L, however preferably at a concentration of about 200 mg/L to about 1000 mg/L. When colchicine is used, microspores are treated preferably at a temperature of about 32° C. for a duration of about 24 hours to about 72 hours. Colchicine may be substituted with other doubling agents, such as amiprophos-methyl, oryzalin, pronamide, trifluralin, etc. As used herein, when referring to chromosome count, “doubling” refers to increasing the chromosome number by a factor of two. For example, a haploid nuclear genome comprising 10 chromosomes is doubled to become a diploid nuclear genome comprising 20 chromosomes. As another example, a diploid nuclear genome comprising 20 chromosomes is doubled to become a tetraploid nuclear genome comprising 40 chromosomes. Confirmation of chromosome doubling can be carried out by flow cytometry or other molecular biology techniques known in the art.

In the DH plant production process, microspore-derived embryos will need to be further converted into plantlets. As illustrated in FIG. 1, there is a ploidy assay step in the standard canola and oilseed rape DH process after plantlet regeneration. The main objective of the ploidy assay is to identify DH plantlets that are doubled, and are therefore suitable for transplanting. This step is beneficial when the doubling efficiency (DE) is low as the majority of plants generated are haploids. For example, if the doubling rate is only 20-30%, then 70-80% of the plants would be haploids that will not set seeds. Unlike plant species such as corn, canola and oilseed rape show no obvious morphology differences between haploids and doubled haploids at the vegetative stage. Therefore, although it is tedious, the ploidy step is needed in the standard canola and oilseed rape DH process.

The novel method of the present invention has increased efficiency at each step of the traditional canola and oilseed rape process. The standard method which only uses colchicine treatment requires 8-16 culture repeats to achieve enough embryos while the new method only requires 1-2 culture attempts. As a result, only 4 donor plants are needed compared to the 24 donor plants needed in the standard method. Furthermore, the embryo production of the standard method is highly variable, where about 50% of the cultures yield few embryos to none at all. The cold-colchicine treatment method described herein has much higher efficiency and consistency in doubled embryo production. As a result, the plantlet ploidy assay step before transplanting to soil can be eliminated using the new process described herein, which saves cost and labor. With fewer repeats, the new process produces a DH population in 7-9 months, which is faster than the 9-12 months typically required using the traditional standard DH production methods.

Furthermore, the cold-colchicine treatment system generally produces more embryos than needed for DH. As an optional step, a subset of embryos may be selected before plant regeneration for a ploidy assay and the calculated doubling efficiency can be used to determine how many embryos to be transferred and how many plantlets to be transplanted at later steps. This new process is much more efficient and cost-effective compared to the standard DH process.

### Definitions

A “late late-uninucleate” (LLU) stage refers to a stage where microspores contain a single nucleus that appears stretched, as opposed to the normal round morphology. At this stage, DNA in the microspore has already duplicated but is still inside one nucleus. This encompasses the late phase of the late-uninucleate stage of microspore development.

An “early bi-cellular” (EBC) stage refers to a stage where microspores comprise two formed nuclei that are bound together and both relatively faint in color and large in size. This stage comprises the first half of the bi-cellular stage.

A “late bi-cellular” (LBC) stage refers to a stage where microspores comprise two nuclei that are clearly separated with the generative nucleus condensed into a much smaller size. This stage comprises the second half of the bi-cellular stage.

The “LLU-EBC” stage refers to microspores at or between the LLU and EBC stages described above.

A “cold-colchicine treatment” refers to cold pretreatment (2-8° C.) of flower buds/microspores for a period, for example 24-48 hours, followed by colchicine treatment of the microspores for a period, for example, 23-72 hours under 32° C.

“Induction media” refers to liquid media containing macro- and micro-nutrients, vitamins, and hormones that is used in microspore culture to induce embryogenesis and development.

An “allele” refers to one or more alternative forms of a genetic sequence; the length of an allele can be as small as 1 nucleotide base. It can also refer to the absence of a sequence. For example, a first allele can occur on one chromosome, while a second allele occurs on the homologous position of a second chromosome, e.g., as occurs for different chromosomes of a heterozygous individual, or between different homozygous or heterozygous individuals in a population. A favorable allele is the allele at a particular locus that confers, or contributes to, an agronomically desirable phenotype, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying disease prone plants. A favorable allelic form of a chromosome site or segment is a chromosome site or segment that includes a nucleotide sequence that contributes to superior agronomic performance at one or more genetic loci physically located on the chromosome interval. “Allele frequency” refers to the frequency (proportion or percentage) at which an allele is present at a locus within an individual, within a line, or within a population of lines. For example, for an allele “A,” diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele. An allele positively correlates with a trait when it is linked to it and when presence of the allele is an indicator that the desired trait or trait form will occur in a plant comprising the allele. An allele negatively correlates with a trait when it is linked to it and when presence of the allele is an indicator that a desired trait or trait form will not occur in a plant comprising the allele.

“Anther culture” is the process of culturing intact anthers.

“Crossed” or “cross” means to produce progeny via fertilization (e.g. cells, embryos, seeds or plants) and includes crosses between plants (sexual) and self-fertilization (selfing).

“Potentiated microspores” refer to those microspores that deviate from the normal microspore maturation process of becoming mature pollen. They adopt a different cell fate and have potential to differentiate into embryos. They often comprise doubled chromosomes but have not undergone cytokinesis. Potentiated microspores may be enriched or obtained by staging and/or specific treatments such as chemical (colchicine), various stresses (heat or cold), etc.

A “doubled haploid or doubled haploid plant or cell”, also referred to as a dihaploid or dihaploid plant or cell, is one that is developed by the doubling of a haploid set of chromosomes. A plant or seed that is obtained from a doubled haploid plant that is selfed any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes. For example, a plant will be considered a doubled haploid plant if it contains viable gametes, even if it is chimeric.

A “microspore-derived embryo” is an embryo that was derived from microspore through tissue culture.

A “doubled microspore-derived embryo” is a microspore-derived embryo that contains 2 sets of homozygous chromosomes.

“Doubled microspore-derived embryogenesis” is a measurement that takes into account of both embryogenesis efficiency and chromosome doubling efficiency. It is calculated by multiplying the total embryos derived from certain number of microspores by the chromosome doubling rate. For example, if there are 2000 embryos derived from 1 million microspores and the chromosome doubling rate is 90%, the doubled microspore-derived embryogenesis is 1800 embryos/million microspores.

“Genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome, or its entire genetic makeup. The terms “phenotype,” or “phenotypic trait” or “trait” refers to one or more traits of an organism. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait.” In other cases, a phenotype is the result of the expression of several genes and their interaction with environments.

As used herein, the term “plurality” refers to more than one. Thus, a “plurality of individuals” refers to at least two individuals. In some embodiments, the term plurality refers to more than half of the whole. For example, in some embodiments a “plurality of a population” refers to more than half the members of that population.

### EXAMPLES

The following examples are included to illustrate embodiments of the invention. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Development of Improved Microspore Staging Technique

In canola/oilseed rape, there is a correlation between flower bud length and microspore developmental stage, however this relationship is influenced by genotype and plant physiology. Selection of buds containing microspores at the correct developmental stage is a critical step in microspore culture. A protocol utilizing DAPI (4′,6-diamidino-2-phenylindole) DNA stain was developed to quickly and precisely identify buds containing microspores at the optimal developmental stage for canola/oilseed rape microspore culture that can be further utilized for both high efficiency embryogenesis and chromosome doubling. This protocol allows for the late uninucleate stage to be further divided into the early late uninucleate (ELU) and late late-uninucleate stages (LLU) Similarly, using this protocol, the bi-cellular stage can be divided into the early bi-cellular (EBC) stage and late bi-cellular (LBC) stage.

All canola or winter oilseed rape inbred or Fi lines that were used in this study are proprietary Bayer Crop Science breeding materials. For canola assays, seeds were sowed and grown in growth chambers with the following set up: Photosynthesis Active Radiation (PAR) at 300 μmol/m²/s—with 16/8 hours light/dark photoperiod, 20° C. during light and 15° C. during dark. When plants started bolting, they were moved to similar light setting conditions but at 12° C./10° C. light/dark for continuous growth. For winter oilseed rape assays, seed sowing and plant growth conditions were the same as those used for canola assays, but included an extra vernalization period when the plants reached the 4-6 leaf stage. Vernalizations were carried out in 4° C. growth chambers, 100 μmol/m²/s for PAR, 12 hours light/dark cycle for 8 weeks. After that, plants were moved to 12° C./10° C. light/dark conditions until young flower buds suitable for microspore culture became available.

Healthy young racemes that were dark green in color and that had a majority of buds that appeared to range in size from 2.0-5.0 mm were harvested from donor plants. Under a dissecting microscope, 1-2 buds measuring 2.5 mm, 3.0 mm, 3.5 mm, 4.0 mm, 4.5 mm, and 5.0 mm were selected from racemes and placed into microcentrifuge tubes containing approximately 0.25 mL fixation solution (3:1 ethanol: glacial acetic acid), such that the buds were completely submerged for at least 15 minutes. The remaining buds/racemes were kept cold until further use. After fixation, 1-4 anthers were dissected from the 2.5 mm buds using a dissecting microscope and placed on a microscope slide that had a small droplet of DAPI (1.5 ug/ml) in the center of the slide. The anthers were gently macerated using a forceps tip in the DAPI solution to release the microspores. Once the anthers had been fully crushed, the slide was covered with a coverslip and sealed. The slide preparation step was repeated for the 3.0 mm, 3.5 mm, 4.0 mm, 4.5 mm, and 5.0 mm buds, accordingly.

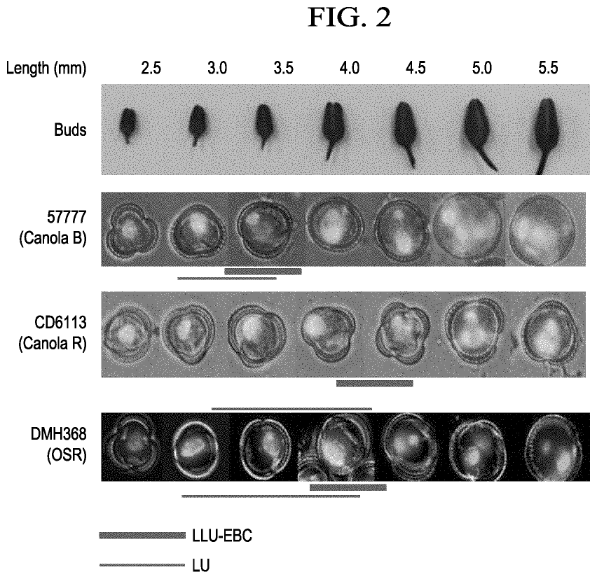

Fluorescence and bright field images of microspores were obtained for each of the bud size groups using a fluorescent microscope. The developmental stage of the microspores from each of the size groups was determined based on nuclear morphology visualized in the images. Based on this determination, buds of a certain size could be expected to contain microspores at or between the LLU and EBC stages and thus bud size can be used as a marker for desired microspore developmental stage (FIG. 2). Buds measuring at lengths that correlate between the latest uninucleate stage (LLU) and the earliest bi-cellular (EBC) stage are preferred. As shown in FIG. 2, the LLU-EBC transition stages are labeled with a thicker bar. In this assay, microspores at the LLU stage contain a single nucleus that appears stretched, as opposed to the normal round morphology. Microspores at the EBC stage comprise two formed nuclei that are bound together and both relatively faint in color and large in size. The correlation between bud length and developmental stage varies based on germplasm background. For example, buds with microspores at the LLU-EBC developmental stage are ˜3.5 mm in length for canola line 57777, while buds with microspores at the LLU-EBC developmental stage are ˜4.0-4.5 mm for canola line CD6113. It was also found that the correlation depends on plant physiology and age. Therefore, it is preferable to do precise staging for each batch of plants prior to culture.

### Example 2: Precise Staging and Specific Colchicine Treatment Parameters Significantly Improves Doubling Efficiency

The LU stage may span a relatively broad range with respect to bud size, depending on the genetic background of the line. Microspores in the LLU-EBC stage are primarily in the G2 or early M stage of the cell cycle (duplication of chromosomes was completed but the cell plate has not yet fully formed). It was hypothesized that microspores in the LLU-EBC stage would have a higher chromosome doubling potential compared to microspores staged as ELU. Given that the new staging protocol enables the visualization of morphology changes within the late uninucleate and bicellular stages, experiments were performed to determine if this narrower developmental stage window would indeed improve the rate of chromosomal doubling in embryos.

Inbred canola line 57777 was used as the microspore donor plant line. Donor plant growth and precise staging were carried out as described in Example 1 above. Buds staged at ELU and LLU-EBC were collected and placed inside a tea strainer. The collected buds were surface sterilized by soaking in 20% Clorox bleach (1.64% Sodium Hypochlorite) for 15 minutes followed by a 1 minute wash using sterile water. This process was repeated 3 times. Surface sterilized buds were then transferred to a 50 ml sterile beaker containing 10 ml NLN13 medium with 0.025 mg/L BAP and placed in cold treatment for 24 hours at 4° C.

After cold treatment, the buds were gently crushed using a pestle to release microspores into the NLN13 medium. The suspension mix was then passed through a 40 μm mesh cell sieve to a 50 ml conical tube and the cell sieve and crushed buds were rinsed with NLN13 to recover more microspores and the total volume was brought to 40 ml. The filtrate was then centrifuged at 250×g for 4 minutes. The supernatant was discarded, and the pellet of microspores was resuspended in 40 ml NLN13BC medium, which contains 13% sucrose, 0.025 mg/L BAP, and 100 mg/L cefotaxime. This washing step was repeated once and the microspores were resuspended in 10 ml NLN13BC medium. Colchicine was added to the microspores at different concentrations (0 mg/L, 200 mg/L, 400 mg/L, 600 mg/L, and 800 mg/L) and incubated for 24 hours at 32° C. After colchicine treatment, microspore cultures were collected and transferred to 50 ml tubes for centrifugation at 250×g for 4 min. The supernatant was decanted, and the microspore pellet was resuspended in fresh NLN13BC medium. The final microspore culture density was adjusted to ˜4×10⁴ counts/ml based on cell counting using a hemocytometer. Approximately 15 mL of the microspore suspension was distributed in 250×100 mm Petri dishes and incubated in the dark at 25° C. for 7-10 days. The cultures were then transferred to a rotary shaker at 50 rpm at 25° C. for 2-3 weeks, which is when generated embryos would reach the cotyledon stage (˜5 mm in length). Embryos at this stage may be used for a ploidy assay or for DH₀ plant regeneration.

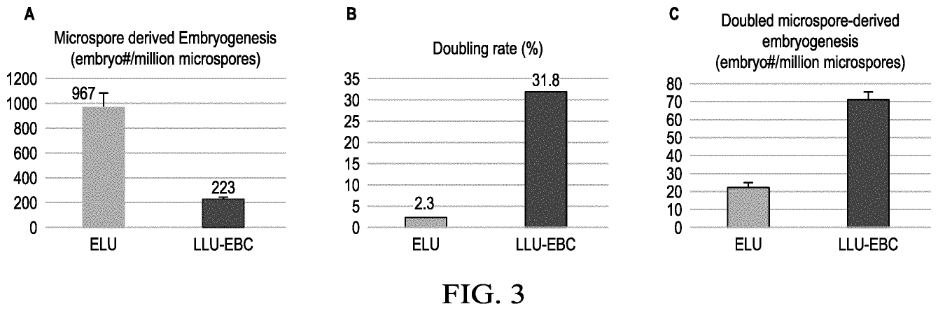

The chromosome doubling rates of embryos that were derived from microspores that were in the ELU and LLU-EBC stages were evaluated. Although the total embryogenesis from ELU staged microspores is much higher than those from LLU-EBC staged microspores (FIG. 3A), a significantly higher doubling rate, i.e. percentage of chromosome doubling, occurred in embryos derived from LLU-EBC stage microspores (FIG. 3B). As a result, the doubled embryogenesis, which is a meaningful metric in breeding practice, was increased approximately 3-fold when using LLU-EBC staged microspores as opposed to ELU staged microspores (FIG. 3C).

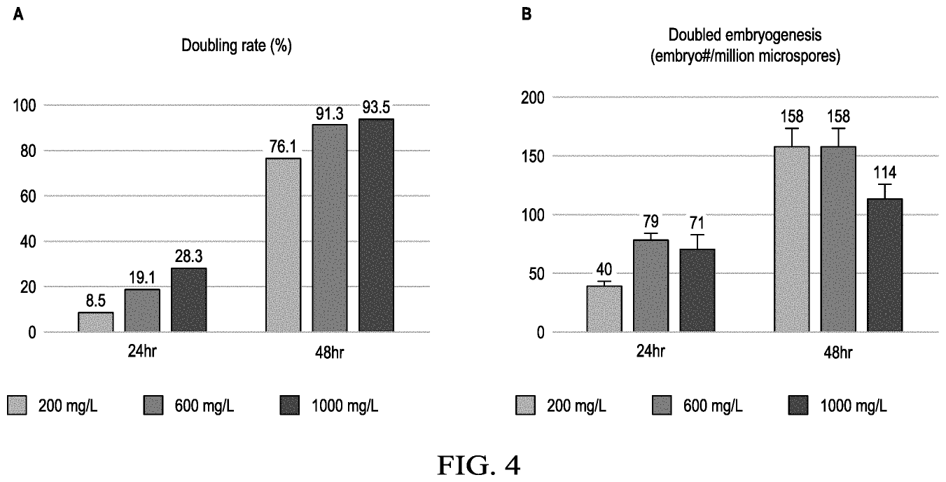

In addition to determining the optimal stage for microspores to increase the doubled embryogenesis rate, the effect of colchicine concentration and duration of treatment on the efficiency of chromosome doubling in embryos was further investigated. Microspore samples were split into 6 treatment groups: 200 mg/L, 600 mg/L, and 1000 mg/L colchicine treatment, each for 24 hours and 48 hours, respectively. It was found that one of the most critical factors in achieving a high chromosome doubling rate is the duration of the colchicine treatment. For all three colchicine concentrations, the treatment duration of 48 hours resulted in a greater than 3×improvement in the chromosome doubling rate when compared to their 24-hour treatment counterpart (FIG. 4A). It was also found that the doubled embryogenesis was significantly increased when the microspores were treated for 48 hours when compared to a 24 hour treatment period (FIG. 4B).

### Example 3: Cold-Colchicine Combination Treatment Leads to a Significant Improvement in Doubled Microspore-Derived Embryo Production

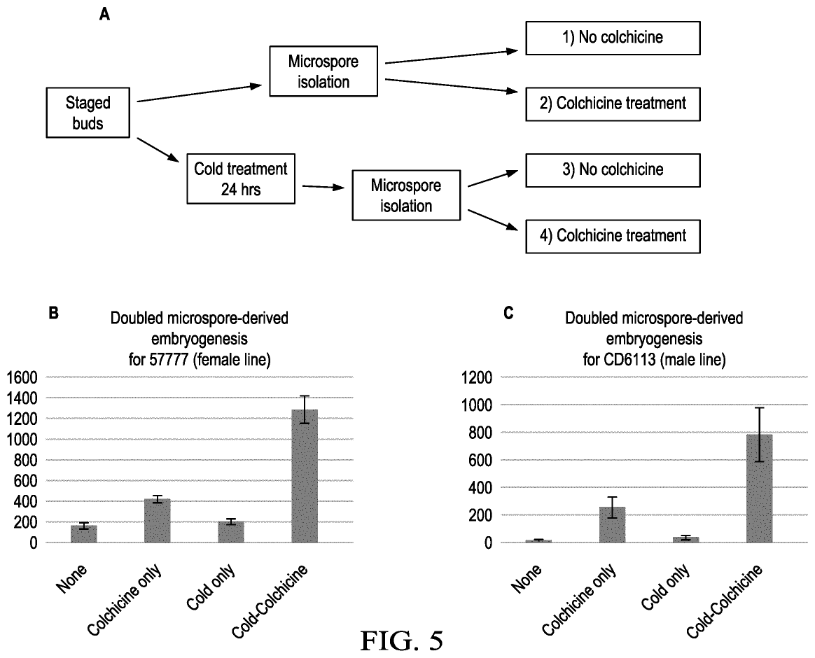

An experiment was performed to determine if utilizing cold pretreated buds followed by colchicine treatment of microspores isolated from those buds would improve the production of doubled embryogenesis in canola comparing to no treatment (Fletcher et al. 1998), or previously reported optimized conditions, such as colchicine treatment only (Szakács and Barnabas, 1995; Zhao et al. 1996; Zhou et al. 2002), or cold treatment only (Lichter 1982; Dunwell et al. 1985; Gu et al. 2004). Canola plants of female line 57777 and male line CD6113 were evaluated for doubled embryo production using the methods described herein. Donor plant growth conditions, bud staging, microspore isolation, and microspore culture were performed as described in Examples 1 and 2 above. Briefly, buds containing microspores at the LLU-EBC stage were pretreated at 2-8° C. for 24 hours. Microspores were isolated from the buds and subsequently treated with colchicine at a concentration of 1000 mg/L for 48 hours at 32° C. After colchicine treatment, the microspores were cultured at 25° C. as previously described. For side by side comparison, a subset of buds was used for microspore isolation and culture the same day, while another subset was pretreated by placing the buds at 2-8° C. for 24 hours. The experimental design process is illustrated in FIG. 5A. The microspores isolated from both cold pretreated and non-pretreated buds were then split into four treatment groups: 1) no cold pretreatment and no colchicine treatment; 2) no cold pretreatment, but treatment with 500 mg/L colchicine for 48 hours at 32° C.; 3) cold pretreatment for 24 hours, but no colchicine treatment; and 4) cold pretreatment for 24 hours and treatment with 500 mg/L colchicine for 48 hours at 32° C. Microspores from buds not pretreated using cold conditions were isolated and treated with colchicine in the same manner as the pretreated buds. After treatment, microspores were washed and cultured using standard procedures. As shown in FIG. 5B and FIG. 5C, cold pretreatment of flower buds followed by colchicine treatment of isolated microspores showed a surprisingly consistent positive effect that led to a significant increase in doubled embryogenesis when compared to previously reported methods. There was a greater than 3-fold increase in doubled embryo production when compared to colchicine-only treatment. The comparison provided herein consists of at least three independent experimental repeats.

Further experiments were performed using a variety of canola and oilseed rape lines that encompassed a wide range of different genetic backgrounds. The control was cold treatment only, which is one the most efficient methods for embryogenesis previously reported. The results are shown in Table 1. When using a cold pretreatment and colchicine treatment combination, the doubled embryo production efficiency across different genetic backgrounds increased ˜4-fold when compared to the production efficiency using cold treatment alone. From the results, it is clear that the cold-colchicine combination delivers a step-change improvement in doubled microspore-derived embryo production.

TABLE 1
Cold-colchicine treatment effect on doubled embryo production
compared to cold only control in different genetic backgrounds.

| | | | # Doubled Embryos | | |
|---|---|---|---|---|---|
| | Genera- | Plant | (embryos/million spores) | | Improve- |
| Line code | tion | type | Cold only | Cold-colchicine | ment |
| 19W0070 | F₁ | OSR | 184 | 456 | 2.47 |
| 19W0091 | F₁ | OSR | 304 | 1568 | 5.15 |
| 19W0572 | F₁ | OSR | 80 | 108 | 1.35 |
| 19W0676 | F₁ | OSR | 174 | 229 | 1.32 |
| 0216BC | F₁ | Canola | 233 | 3306 | 14.18 |
| 0386W | F₁ | Canola | 121 | 430 | 3.56 |
| Average | | | | | 4.67 |

1. A method for producing embryos from microspores comprising the steps of:
a) obtaining a plurality of flower buds from a donor plant;
b) determining the developmental stage of microspores comprised within said flower buds;
c) selecting flower buds comprising microspores at a desired developmental stage;
d) treating said flower buds;
e) isolating microspores from said flower buds; and
f) culturing said microspores in induction medium, thereby producing embryos.

2. The method of claim 1, wherein:
a) the desired developmental stage is defined as the late late-uninucleate stage or the early bi-cellular stage;
b) said treating comprises incubating said flower buds at a temperature of about 2° C. to about 8° C. for about 24 hours to about 72 hours;
c) said culturing is carried out at a density of about 2.0×10⁴ to about 1.0×10⁵ microspores/mL; or
d) said donor plant is a canola, cauliflower, broccoli, pepper, cabbage, soybean, cotton, or corn plant.

3. The method of claim 2, wherein:
a) said developmental stage is determined by nuclear staining of said microspores;
b) said incubating is carried out for about 24 hours to about 48 hours; or
c) said culturing is carried out at a density of about 4.0×10⁴ microspores/mL.

4. (canceled)

5. (canceled)

6. (canceled)

7. (canceled)

8. (canceled)

9. A method for producing potentiated microspores comprising the steps of:
a) obtaining a plurality of flower buds from a donor plant;
b) determining the developmental stage of microspores comprised within said flower buds;
c) selecting flower buds comprising microspores at a desired developmental stage;
d) pretreating said flower buds;
e) isolating microspores from said flower buds; and
f) treating said isolated microspores with a chromosome doubling agent to produce potentiated micro spores.

10. The method of claim 9, wherein:
a) the desired developmental stage is defined as the late late-uninucleate stage or the early bi-cellular stage;
b) said pretreating comprises incubating said flower buds at a temperature of about 2° C. to about 8° C. for about 24 hours to about 72 hours;
c) said treating is carried out at a density of about 2.0×10⁴ to about 1.0×10⁵ microspores/mL;
d) the chromosome doubling agent is colchicine;
e) said treating comprises incubating said microspores at a temperature of about 32° C. for a duration of about 24 hours to about 72 hours; or
f) said donor plant is a canola, cauliflower, broccoli, pepper, cabbage, soybean, cotton, or corn plant.

11. The method of claim 10, wherein:
a) said developmental stage is determined by nuclear staining of said microspores;
b) said incubating is carried out for about 24 hours to about 48 hours;
c) said treating is carried out at a density of about 4.0×10⁴ microspores/mL; or
d) the colchicine is at a concentration of about 25 mg/L to about 1600 mg/L.

12. (canceled)

13. (canceled)

14. (canceled)

15. (canceled)

16. (canceled)

17. (canceled)

18. The method of claim 11, wherein said treating comprises applying colchicine at a concentration of about 100 mg/L to about 1000 mg/L.

19. The method of claim 18, wherein said treating comprises applying colchicine at concentration of about 500 mg/L to about 1000 mg/L.

20. (canceled)

21. The method of claim 10, wherein the incubating is carried out for about 40 hours to about 48 hours.

22. (canceled)

23. The method of claim 9, wherein said method further comprises:
g) culturing said microspores to obtain at least a first embryo; and
h) regenerating a doubled haploid plant from said embryo.

24. A method for producing doubled haploid embryos from micro spores comprising the steps of:
a) providing microspores at a desired developmental stage;
b) pretreating said microspores under cold conditions for a fixed period of time;
c) treating said microspores in a medium containing an effective concentration of colchicine to induce chromosome doubling;
d) culturing said treated microspores of step c) in induction medium, thereby producing embryos; and
e) recovering doubled haploid embryos from said induction medium.

25. The method of claim 24, wherein:
a) said cold conditions comprises a temperature of about 0° C. to about 25° C.;
b) said fixed period of time is about 12 hours to about 72 hours;
c) said effective concentration of colchicine is about 25 mg/L to about 1600 mg/L in the medium; or
d) step c) is carried out at a fixed temperature for a fixed period of time.

26. The method of claim 25, wherein:
a) said cold conditions comprises a temperature of about 2° C. to about 8° C.;
b) said effective concentration of colchicine is about 200 mg/L to about 1000 mg/L in the medium; or
c) step c) is carried out at 32° C. for about 12 hours to about 72 hours.

27. (canceled)

28. (canceled)

29. (canceled)

30. (canceled)

31. (canceled)

32. A method for producing doubled haploid Brassica embryos comprising the steps of:
a) providing Brassica microspores at or between the late late-uninucleate and early bi-cellular developmental stages;
b) pretreating said microspores under cold conditions for about 12 hours to about 72 hours;
c) treating said microspores in a medium containing an effective concentration of colchicine to induce chromosome doubling;
d) culturing said treated microspores of step c) in induction medium, thereby producing embryos; and
e) recovering doubled haploid Brassica embryos from the induction medium.

33. The method of claim 32, wherein:
a) said cold conditions comprises a temperature of about 2° C. to about 8° C.; or
b) said effective concentration of colchicine to induce chromosome doubling is about 25 mg/L to about 1600 mg/L in the medium.

34. (canceled)

35. The method of claim 33, wherein said effective concentration of colchicine to induce chromosome doubling is about 200 mg/L to about 1000 mg/L in the medium.
